(19) United States
(12) Patent Application Publication
(10) Pub. No.: US 2016/0215249 A1
(43) Pub. Date: Jul. 28, 2016

(54) DISPOSABLE BIO-REACTOR SYSTEM

(71) Applicants: Ravindranath Gandlur, Hoskote (IN); Prasad Rao Gandlur, Narayanapura Bangalore (IN)

(72) Inventors: Ravindranath Gandlur, Hoskote (IN); Prasad Rao Gandlur, Narayanapura Bangalore (IN)

(21) Appl. No.: 15/006,919

(22) Filed: Jan. 26, 2016

Related U.S. Application Data
(63) Continuation of application No. 12/922,690, filed on Sep. 15, 2010.

(51) Int. Cl. C12M 1/00; C12M 3/00

(52) U.S. Cl. CPC C12M 23/28; C12M 23/26; C12M 21/08; C12M 23/14

(57) ABSTRACT

A disposable bioreactor system with a focus on improving cell culture technology by introducing a disposable bioreactor bag which operates or functions in conjunction with it unique mechanism of external non-rotating gyrating motion that uses gravity in a natural way to make the fluid flow circumferentially in it. The bioreactor is eminently suitable to won a variety of living cells and has the advantage of tangential flow to avoid clogging of filters.

### CROSS REFERENCE TO RELATED APPLICATIONS

This specification is the complete specification of the provisional Patent Application No. 2836/Che/2008 dated 18.11.2008 filed by Ravindranath Gandlur and Prasada Rao Gandlur.

### FIELD OF INVENTION

This invention relates generally to the field of disposable bioreactors. The invention is a novel disposable bioreactor apparatus with a focus on improving cell culture technology by introducing a disposable bioreactor bag which operates or functions in conjunction with a unique mechanism of external non-rotating gyrating motion that uses gravity in a natural way to make the fluid flow circumferentially in it.

### BACKGROUND OF INVENTION

There is a large amount of activity in the general field of bioreactors and bioreacting devices culture systems for growing bacterial, fungal, mammalian, insect, plant or any other living cell types for studies related to growth and other characteristics, for production of metabolites, antibodies, proteins, viruses, polysaccharides or appropriate components and derivative of such cells in culture in closed or semi-closed reactor systems in batch systems, or fed batch system or continuous culture systems, including perfusion systems. The prior art is flooded with a large number of patented inventions and technical literature on the subject in question.

It is common knowledge today that cultivation of cells in small, medium and large scale in bioreactors, euphemistically called fermenters is traditionally carried out in rigid glass or stainless steel vessels with built in mechanism of agitation by stirring mechanically or by air. The vessels have cylindrical shapes with round or flat bottom. The impellers used are of different shapes and the speeds of stirring range from a few RPH (revolutions per hour) to hundreds of RPM (revolutions per minute). However, the efficiency of aeration and keeping the cells in proper suspension especially in larger cell types such as animal cell cultures is a challenge since the cells either tend to settle down or they collide with the stirrer or among themselves. Besides, these designs employ in situ cleaning and sterilization by steaming which is time consuming, laborious, and expensive for proper validation.

It is noteworthy to point out that gradually the emphasis and focus, has shifted to the disposable type pre-sterilized bioreactors to overcome not only the inherent disadvantages occurring in the conventional stainless steel re-usable type bioreactors but also to address the concerns of the cost implication involved. Prior art (e.g. US Patent Application 2005/023,9198) lists some disposable bioreactors with internal stirring mechanisms using impellers. These disposable bioreactors suffer from the disadvantage that the stirring is invasive and is likely to harm the cells under suspension. In the case of cells growing on micro-carriers such stirring may produce large shear forces that dislodge the cells from the micro-carriers and may even cause damage to the cells. Also, the internal stirring mechanism renders them less cost effective.

The disposable bioreactors that use external stirring of containers by rocking motion may be exposed to collisions among the suspended cells when the flow continuously changes direction. The U.S. Pat. No. 6,544,788 issued in favor of Vijay Singh discloses an invention pertaining a disposable bioreactor for perfusion cell culture. Cells are grown in a plastic bag that is rocked and aerated on a mechanical platform. This invention uses rocking motion in a single axis to achieve the mixing of the contents.

In these disposable bioreactors there are invariably dependent areas i.e. lowest part of the vessel where sedimentation may occur and the stirring may fail to keep the cells in suspension which may be detrimental to the cells and may reflect in poorer yields of the expected products. In these dependent areas where stirring is inefficient the conditions of the culture medium such as pH may not be uniform.

The disposable bioreactors are also used as perfusion systems wherein the liquid from the bioreactors is pumped out using a filter to retain cells in the container. This mechanism suffers from clogging of filters if the filtration is of cross flow type. Prior art (U.S. Pat. No. 6,544,788) discloses some attempts to prevent the clogging of filters while harvesting the contents by incorporating filters that move over the surface of the culture medium taking advantage of the tangential motion.

As can be seen, the prior art profile indicates the existence of numerous disposable bioreactors and the agitation mechanisms. But a bioreactor of disposable type which functions on the unique concept of a specially designed platform with a central elevation or protrusion that would convert any flexible disposable bag or sac or container suitable for use with the platform which has a natural tilt with the horizontal to take advantage of gravity for movement of the bag contents, which coupled with a gyratory or swaying motion of sequential lowering of the platform along the circumference, imparting a continuous unidirectional flow to the contents of the disposable bioreactor bag is hitherto unknown and would be a novel improvement to the state of the art. Also the concept of such a unidirectional circumferential flow over a filter placed inside the bag in contact with the contents resulting in a tangential flow across the filters to prevent clogging of the filters is also not known in the art and would be a novel improvement to the state of the art.

In view of the above mentioned problems of the prior art, the objectives of the present invention are:
1. to provide combination of convenience and utility and an improved performance.
2. to include all the advantages of the prior art,
3. to attempt to overcome the major disadvantages/drawbacks of the prior art, and
4. to provide a novel disposable bioreactor apparatus, a disposable bioreactor bag and a whole system which can be used for a wide gamut of applications, for wider range of cell cultures.

### SUMMARY OF INVENTION

The present invention offers to improve cell culture technology by introducing a novel disposable bioreactor system consisting of a disposable bag, a specially designed platform with a central elevation or protrusion that would virtually convert any flexible disposable bag that is placed on it into an annulate structure, the platform having a natural tilt with the horizontal to take advantage of gravity for movement of the bag contents and a unique mechanism of external non-rotating gyratory or swaying motion to impart unidirectional circumferential flow to the contents of the bioreactor bag. The platform does not rotate but every point on the circumference is lowered (to a variable degree) sequentially starting from 0 deg position to 360 deg position in a continuous motion so that the contents of the bag move by gravity in a unidirectional, circumferential motion. This is achieved by a specially designed gyrating mechanism.

For a better understanding of the invention, its operating advantages and the specific objects attained by its user, reference should be made to the accompanying drawings and descriptive matter in which there are illustrated embodiments of the invention.

### BRIEF DESCRIPTION OF THE DRAWINGS

In these drawings wherever the view of the bag is shown in horizontal position is done for the purpose of understanding the invention, whereas it is inclined under normal operational conditions.

FIG. 1. shows the top and sectional front views of a type I bag (1A) which is a circular bag with a central hole. The view depicts the bag filled with air

FIG. 2. shows the top and front views of a type II bag (1B) which is a plain circular bag without the central hole. The view depicts the bag filled with air

FIG. 3. shows the type I bag (1A) placed on top plate of platform (2) with the culture media filled, depicting the basket support (14) and the cylindrical central support (13A).

FIG. 4. shows the type II bag (1B) placed on top plate of the platform (2) with the culture media filled wherein the conical central support (13B) along with the basket support (14) that serves to provide an annular cavity for the contents of the bag.

FIG. 5 shows the type I bag (1A) on the top plate of platform (2) with the natural inclination that provides the movement of the bag contents to one end by gravity.

FIG. 6 show the type II bag (1B) on the top plate of platform (2) with the natural inclination that provides the movement of the bag contents to one end by gravity.

FIG. 7 depicts the stages of the gyratory motion of the top plate of platform (2).

FIG. 8 shows the picture of the type I bag (1A) with the inlet and outlet ports consisting of a tube (8) going through a threaded neck (9) or a flanged bush (10) welded to the bag (1A).

FIG. 9 shows one embodiment of the type I bag (1A) with a filter (7) placed in between the top and bottom layers to be used for perfusion cultures.

FIG. 10 shows the sectional view of the bag (1A) and filter (7).

FIG. 11 shows the schematic of the gyratory mechanism.

FIG. 12 shows one embodiment of the bioreactor system enclosed in an incubator hood (15) for temperature control.

### DETAILED DESCRIPTION OF INVENTION

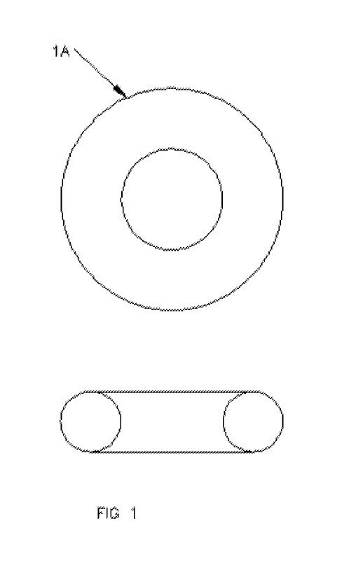

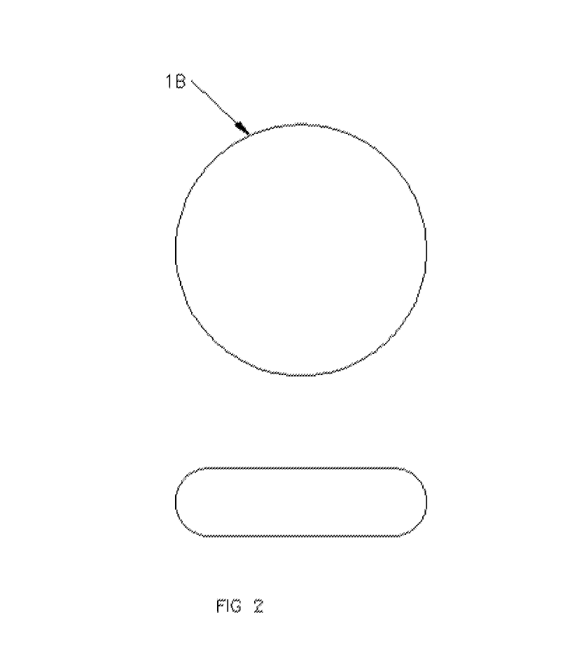

The invention is a disposable bioreactor bag that is closed, sealed and pre-sterilized. This bag(1A or 1B) as shown in FIG. 1 and FIG. 2 is made of soft or hard plastic material such as polyvinyl chloride, polyethylene or any other polymer approved for human medical use. This bioreactor bag has a round shape with or without a central hole, and on being placed on the specially shaped platform the bag would essentially form an annular inner chamber holding the contents.

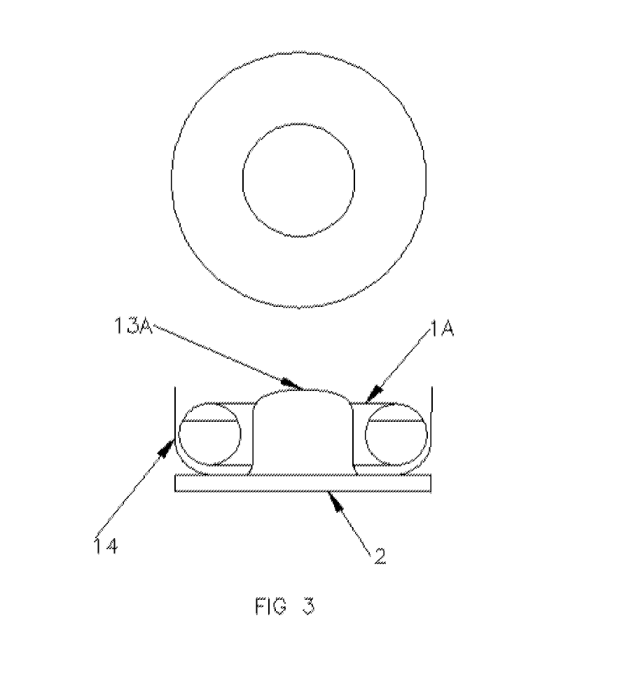

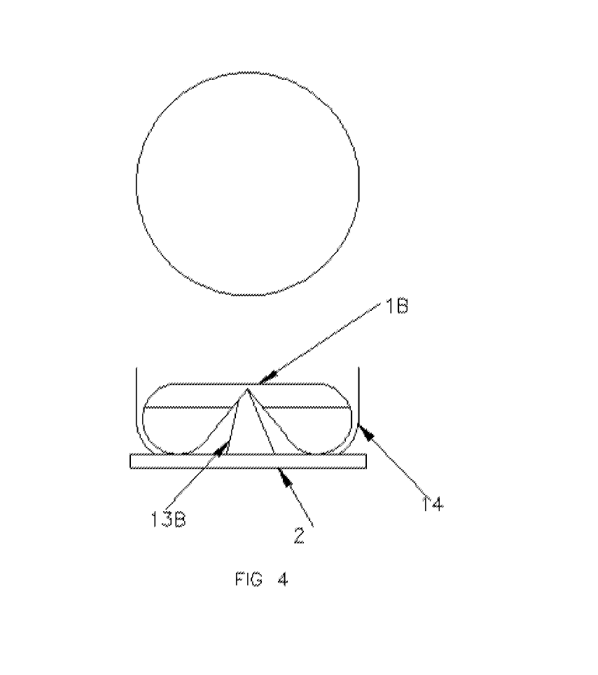

This is ensured by the cylindrical central support (13A) and basket support (14) in the case of bag (1A) and conical central support (13B) and the basket support (14) in the case of bag (1B) as shown in FIG. 3 and FIG. 4. Clean filtered air is pumped into the inner chamber of the bag to sufficiently inflate and provide the shape and rigidity to the bag when placed on the specially shaped platform.

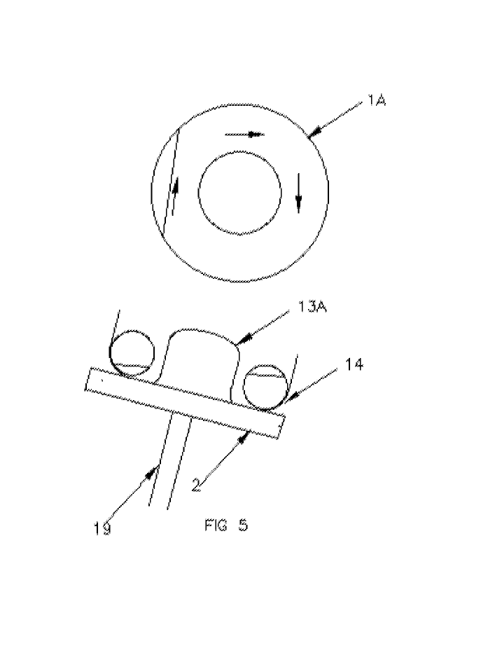

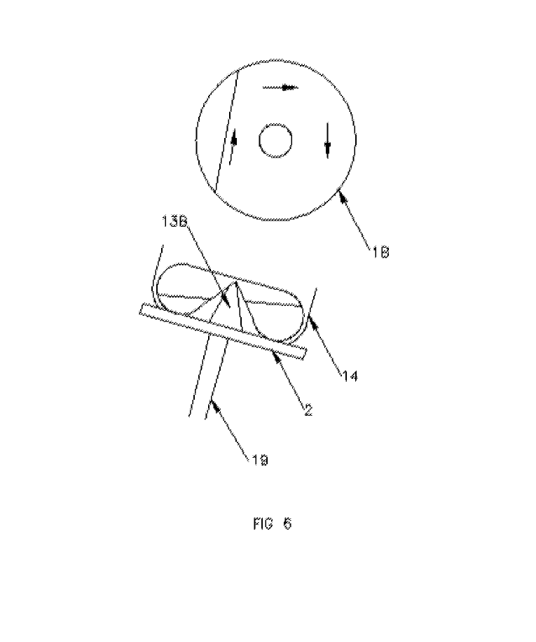

The platform has a natural inclination with reference to the horizontal under normal operating conditions and the contents of the bag move by gravity to one end of the bag as shown at FIG. 5 and FIG. 6.

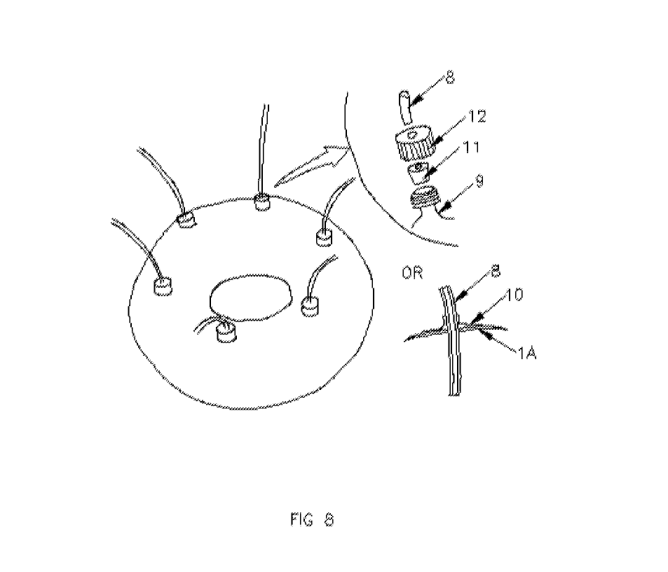

The bag has inlet and outlet ports by means of tubes (8) going through silicone plug (11) and cap (12) in case of threaded type (9) port or tubes (8) welded to flanged bush (10) in case of flanged bush type (10) port for delivering ingredients into the culture system and to enable taking samples or harvests of the contents as shown in FIG. 8. The bag is provided with ports of either option or a combination of both.

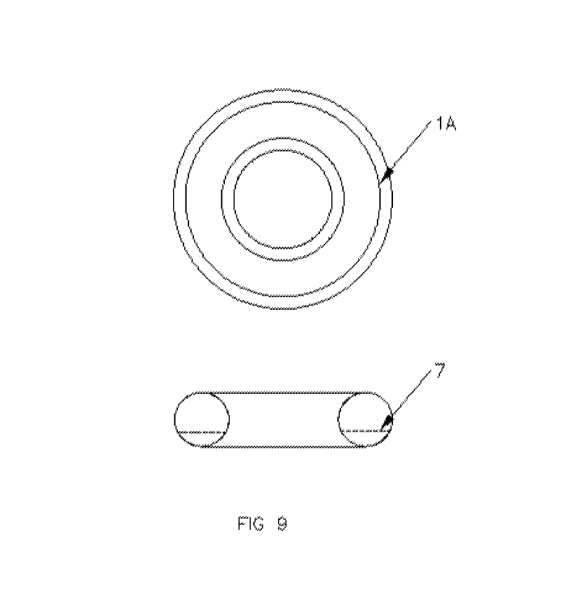

A largely porous septum partition or filter (7) inside the bag in some of the intended units, divides the inner chamber into two or more partitions so that in one partition could be placed the culture medium and the other chambers could be used for placing micro or macro carrier beads, pellets or fibers. The septal partition could be in the form of meshes or nets or hydrophilic filters so that the contents of the disposable bags could be sampled either whole or without the cells and other ingredients. FIG. 9 depicts one such placement of a filter (7) in the bag (1A) for use as a perfusion system.

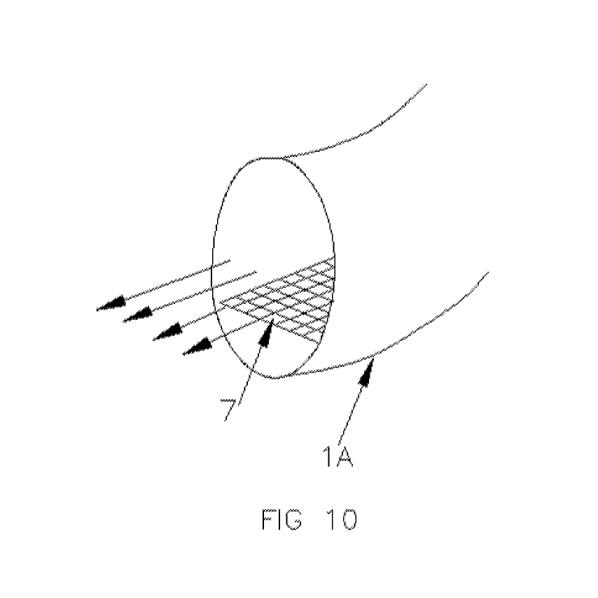

The tangential flow of the medium over the filters would prevent clogging of the filters as the fluid moves on top of the filter (7) in a tangential fashion removing any debris as it forms. This is shown in FIG. 10.

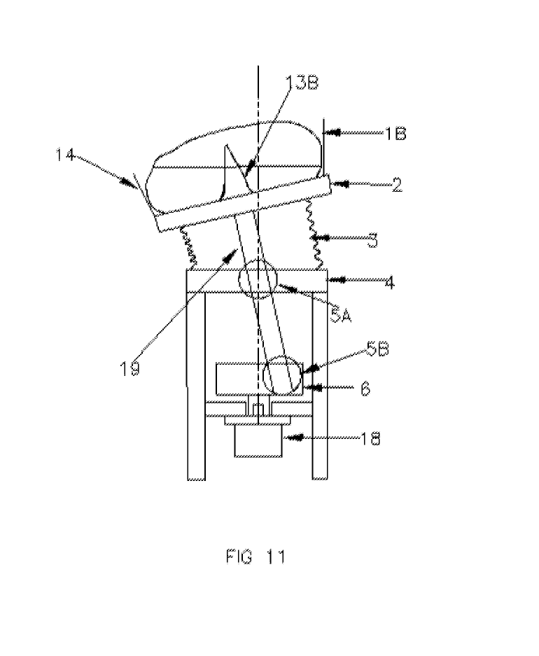

A schematic of the gyratory mechanism is shown at FIG. 11. The top plate of platform (2) is fixed to the main shaft (19) which passes through two spherical bearings (5A and 5B). The pivoting bearing (5A) is fixed to the stand (4) and the rotating bearing (5B) is fixed to the eccentric shaft (6). The eccentric shaft (6) is connected to a variable speed electric motor (18). As the motor (18) rotates the. eccentric shaft (6), the bearing end of the main shaft (19) rotates eccentrically thus providing an angular rotation to the other end fixed to the platform (2). The rotation of top plate of platform (2) is arrested by means of the tensioner springs (3) which are fixed to the stand. Because of this the top plate of platform (2) has no rotational motion but is forced to gyrate. This non-rotational angular gyration causes sequential lowering of the platform around its periphery. As the disposable bioreactor bag (1A or 1B) is held on to the top plate of platform (2) by means of central support (13A or 13B) and the basket support (14), this gyratory motion imparts a continuous unidirectional flow to the contents of the disposable bioreactor bag.

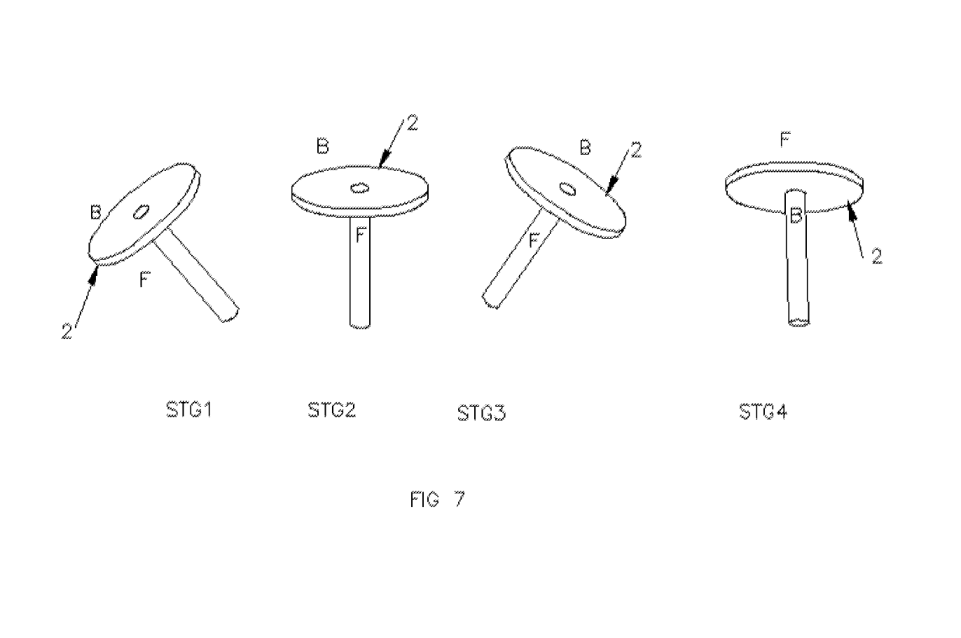

FIG. 7 shows the views of 4 stages of the gyratory motion. The letters F and B denote the front and back of the platform for a person standing in front of the platform (2) The stages 1 to 4 depict the sequence of gyration starting from stg 1 in which the platform (2) is shown tilted to the left of the viewer to tilting towards the viewer at stg 2, then to the right of the viewer at stg 3 and then to the tilting away from the viewer at stg 4 after which one cycle would be completed when it returns to stg 1.

While the liquid levels at any point of the cross sectional view of the annular chamber vary from 20% to 80% due to the swaying motion, it also imparts a gentle sideways movement to the suspended particles in the liquid, so as to keep the cells under constant flotation.

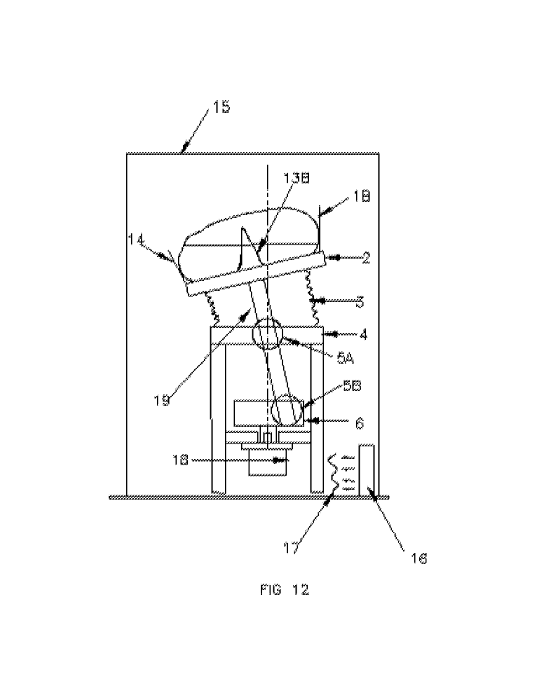

In one embodiment, as shown in FIG. 12, the entire system (disposable bioreactor and the gyrating platform) is enclosed in a transparent polycarbonate chamber (15) with appropriately placed air circulating fan (16), heater with temperature control (17) to maintain the cultures at required set temperature. As the temperature could be set from 20° C. to about 40° C. and the rate of gyration from a few cycles per hour to a few hundred cycles per minute, a very efficient mixing of the contents and gaseous exchange takes place as there are no dead spaces in the Bioreactor bag.

This invention, therefore, is eminently suitable for culturing and growing bacterial and mycotic cells, insect, avian and mammalian cells, in suspension or in the case of anchorage dependent cells, on microcarriers or macro carriers as in perfusion systems, for products of cell culture such as monoclonal antibodies, DNA and its derivatives, genetically cloned proteins, biotherapeutic agents and vaccines.

The exemplary embodiments described here in detail are for illustrative purposes and are subject to numerous variations. It is understood that various omissions, substitutions or equivalents are contemplated as circumstances may suggest or render expedient, but is intended to cover the application or implementation without departing from the spirit or scope of the invention. Accordingly, the invention is intended to embrace all such alterations, modifications and variations as may fall within the spirit and scope of the present invention.

### EXAMPLE 1

### Pilot Scale Cultivation of Mammalian Cells

Material and Methods: A Disposable Bag having a total internal capacity of 7.2 liters with a cross sectional diameter of 4 inches and an outside dia of 18 inches was used. 2.0 liters of MEM Joklik Modification with Earles salts, and 1-Glutamine (HIMEDIA Cat.AT079) with 10% Foetal Bovine Serum and 2.0 gms per liter sodium bicarbonate, filter sterilized by 47 mm ss Filter Holder using Millipore 0.22□, 0.45□ and Depth Glass prefilter. The bag is pressurized with filtered air using a small blower pump to give approximately 0.2 bar. BHK cells were harvested from confluent sheets, trypsinized and adopted to the spinner medium without Ca++ and Mg++ in a 300 ml glass spinner flask for 36 hours were transferred into the Disposable Bag. A total of 50 million cells were transferred into the disposable bag. The flow rate was adjusted to approximately 600 ml per second by adjusting the speed of rotation of the platform (20 rotations per minute). Samples were collected at 0 hrs and subsequently 12 hour intervals after the first 24 hrs.

Results: Samples were tested for 1. Sterility 2. pH 3. Cell count (total and viable count by 0.1% trypan blue staining).

Sterility of samples was tested by
1. Incubating 2 ml of the sample at 37° C.
2. By inoculation of 0.5 ml in 12 ml of Alternate Thioglycollate medium in a 20 ml screw cap tube and incubating at 37° C.
3. By inoculating 0.5 ml in Soyabean Casein Digest Medium in a 120×15 mm test tube with a cotton stopper.

All the samples collected were sterile.

The pH and Cell counts are shown in the following the table.

| In hrs | 0 | 24 | 36 | 48 | 60 |
|---|---|---|---|---|---|
| Total cells | 30000 | 30000 | 34000 | 48000 | 120000 |
| (/ml) | | | | | |
| Dead cells/ | — | — | | — | — |
| ml | | | | | |
| In hrs | 72 | 84 | 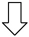 96 | 108 | 120 |
| Total cells | 128000 | 250000 | 600000 | 1500000 | 4000000 |
| (/ml) | | | | | |
| Dead cells/ | — | — | few | few | few |
| ml | | | | | |
| In hrs | 132 | 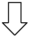 144 | 156 | 168 | 180 |
| Total cells | 4300000 | 7000000 | 7100000 | 9000000 | 11200000 |
| (/ml) | | | | | |
| Dead cells/ | few | 4.6 × 10⁵ | 4.0 × 10⁵ | few | 5.0 × 10⁵ |
| ml | | | | | |
| In hrs | 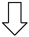 192 | 204 | 216 | 228 | 240 |
| Total cells | 16000000 | 15000000 | 13800000 | 15300000 | 21000000 |
| (/ml) | | | | | |
| Dead cells/ | 1 × 10⁶ | 2.5 × 10⁶ | 3 × 10⁶ | 3 × 10⁶ | 4.1 × 10⁶ |
| ml | | | | | |

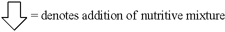

In a normal stationary bottle, the cell density of BHK cells increases approximately 20 to 40 fold.

The limit is generally due apart from contact inhibition, to density dependent inhibition because of the depletion of nutrients especially glucose, I glutamine and vitamins. The growth patterns seen in the disposable bioreactor are similar and further studies by replenishing the nutritional components will be of interest.

### EXAMPLE 2

### Culture of Saccharomyces Boulardii

A culture bag of 2.5 working volume which is presterilized by gamma irradiation is taken. YEPD Medium (Reagent Card No. Lablinks R0007 dt 7 Jun. 2009.) 2.0 lit transferred to sterile bag under sterile precautions. An overnight culture of 20 ml of S boulardi seed was inoculated. Stirring set at 20 RPM initially and increased after 24 hours. Total cultivation time was 78 hrs.

Seeding count was approx 8000 million organisms. At the end of the culture period, a total count of approx. 500 million organism per ml (total quantity 2 l) Total cell yield was roughly 1000000 million organism or approximately 120 times the initial inoculum.

The exemplary embodiments described herein detail for illustrative purposes are subject to numerous variations. It is understood that various omissions, substitutions or equivalents are contemplated as circumstances may suggest or render expedient, but is intended to cover the application or implementation without departing from the spirit or scope of the invention. Accordingly, the invention is intended to embrace all such alterations, modifications and variations as may fall within the spirit and scope of the present invention and is limited only by the claims made herein.

We claim:

1. A disposable bioreactor system suitable for culturing a variety of living cells comprising,
(i) a disposable pre-sterilized bioreactor container which has a circular shape;
(ii) a gyrating platform on top of which the bioreactor container is placed;
(iii) means to hold the bioreactor top of the gyrating platform;
(iv) means to produce gyratory motion by providing a tilt to the top plate of said gyrating platform with reference to the horizontal plane by at least 1 degree.
(v) means to produce gyratory motion by sequentially lowering the periphery of said top plate to the angle of tilt along the periphery in a continuous gyratory motion;
(vi) means to adjust the speed of the said gyratory motion;
(vii) means to introduce material into the said disposable bag, in the form of inlet ports;
(viii) means to remove material from the said disposable bag, in the form of outlet ports; and
(ix) ports for sensors.

2. The disposable bag of claim 1 wherein the material of construction is of soft flexible plastic sheets such as medical grade polyvinyl chloride or polyethylene sheets or any other polymeric material such that the inner chamber of the said bioreactor can withstand a pressure of 1 bar.

3. The bioreactor bag of claim 1 is welded in such a way that when inflated, the bag attains the shape of a doughnut or a circular pillow creating an inner chamber in which culture media can be introduced.

4. Ports for sensors according to claim 1 provide the means to introduce sensors to said bioreactor to measure temperature, pH and pO₂ thorough specially designed ports.

5. Culture media and inoculums are introduced into the bioreactor of claim 1 through inlet ports, said inlet ports being connected by means of inlet pipes to the source of culture media and inoculums.

6. Gaseous material is introduced into the bioreactor of claim 1 through inlet ports, said inlet ports being connected by means of inlet pipes to the source of gaseous material.

7. The disposable bioreactor bag of claim 1 wherein the material harvesting and gas vents are in the form of outlet ports with the said outlet ports being connected by means of outlet pipes, said outlet ports suitably located to act as the means to collect the contents from the bottom of said bio reacting container either through filters or without filters.

8. The top of the gyrating platform of claim 1 is a circular top plate.

9. Holding means according to claim 1, is a supporting basket.

10. Holding means according to claim 1, said holding means securely holding the bioreactor to the top of the said gyrating platform, are at least two clamps.

11. The disposable bioreactor system of claim 1, in one preferred embodiment, has means to recirculate the drawn contents back into the bag through a reservoir for perfusion cell cultures and also means to maintain the levels of the bag contents.

12. The disposable bioreactor system of claim 1, in one preferred embodiment, has a hood made of clear plastic with temperature control and air circulation in which the said system is placed to maintain the temperature for cell cultures.

13. The disposable bioreactor bag of claim 1 with all the ports and accessories, is pre-sterilized by steam or gamma radiation or ethylene oxide in a double sealed package to ensure sterility.

14. Means to provide gyratory motion to the platform according to claim 1 include provision to tilt the top plate with reference to the horizontal plane in a preferred embodiment in a preferred range of 5 to 30 degrees such that the contents of a bag placed on such a platform move in the direction of the tilt by gravity.

15. Means to provide gyratory motion to the top plate of the platform according to claim 1, includes provision to sequentially lower the periphery continuously through a variable speed electrical motor so that the contents of a bag placed on such platform attain a unidirectional circumferential flow aided by gravity.

16. Means to provide gyratory motion as in claim 1 comprising a main shaft passing through a pivoting spherical bearing and a rotating spherical bearing which is eccentrically rotated by means of a variable speed electric motor and the rotation of the platform prevented by tensioner springs resulting in a non-rotational gyratory motion.

17. The gyrating platform according to claim 1 will have, in one preferred embodiment, a central supporting protrusion or elevation on the top plate in the form of a cylinder or a cone.

18. The central supporting protrusion or elevation according to claim 17 shaped as a cone is designed such that any plain bag having the shape of a plain pillow, when placed on the top plate of said gyrating platform forms an annulus and allows the contents to move in a unidirectional flow due to the gyratory motion.

19. The central supporting protrusion or elevation according to claim 17 shaped as a cylinder is designed such that a bag having the shape of a doughnut, when placed on the top plate of said gyrating platform is located and supported by the protrusion.

20. The disposable bioreactor bag of claim 1, has in one embodiment, a septum partition that divides the inner chamber into one or more parts to accommodate soft or hard, micro or macro carrier beads, pellets or glass or plastic fibres that provide the surface for anchorage dependent cell cultures.

21. The disposable bioreactor bag of claim 20, wherein said septum partition may be made of sieves, meshes or filters to retain the micro or macro carrier beads and also to aid in gas exchange.

22. The disposable bioreactor bag of claim 20, wherein the said septum partition, made up of a filter element is placed such that the flow of the bag contents over the filter result in a tangential motion preventing clogging of the filter.

23. The disposable bioreactor apparatus of claim 20, wherein said macro carrier beads, pellets or glass/plastic fibers are provided for ensuring the availability of a surface for anchorage and growth of certain cells.

24. The anchorage material according to claim 23, could be constituted from any known synthetic polymers either used in isolation or in combination with each other.

25. The anchorage material according to claim 23 could be treated or coated with certain substances such as proteins or ionic charges to improve the plating efficiency of the anchorage dependent cell cultures.
